(19) United States
(12) Patent Application Publication
(10) Pub. No.: US 2009/0041690 A1
(43) Pub. Date: Feb. 12, 2009

(54) AUTOBRONZING COMPOSITION

(76) Inventors: James Wilmott, Clinton, NJ (US); Carolyn Romaine, Somerset, NJ (US)

Correspondence Address: YOUNG & THOMPSON, 209 Madison Street, Suite 500, ALEXANDRIA, VA, 22314, US

(21) Appl. No.: 12/280,796

(22) PCT Filed: Feb. 27, 2007
(86) PCT No.: PCT/IB07/00473

§ 371 Date: Sep. 25, 2008

Related U.S. Application Data
(60) Provisional application No. 60/777,117, filed on Feb. 28, 2006.

(51) Int. Cl. A61K 8/72; A61Q 19/04

(52) U.S. Cl. 424/59

(57) ABSTRACT

The present invention pertains to an auto-bronzing composition including (i) at least one auto-bronzing agent, (ii) at least one auto-bronzing reactive material (“ARM”) chosen from the group consisting of amphiphilic copolymers of AMPS with at least one hydrophobic comonomer and (iii) at least one specific emulsifier. The emulsifier (iii) is preferably PEQ-100 stearate, glyceryl stearate, steareth-2 or steareth-21, or mixtures thereof. The emulsifier stabilises the ARM. It also pertains to the cosmetic use of this auto-bronzing composition for artificially tanning skin.

### FIELD OF INVENTION

The present invention relates to topical autobronzing compositions. More particularly, the autobronzing compositions of the present invention have improved onset, duration, and quality of tan without a decreased rate of loss of the autobronzing active agent and no or reduced off-odor.

### BACKGROUND

Beginning in about the 1920s, sunbathing and tanned appearance became very fashionable. Since that time, tanned skin has, and continues to be, highly desired. Around the same time, in the 1920s, the skin-coloring properties of dihydroxyacetone (“DHA”) were discovered. It is now well-known in the art that DHA reacts chemically with amino acids in the proteins on the skin's surface, resulting in coloration similar to that produced by melanin from exposure to ultraviolet radiation (“UVR”). In general, DHA-containing topical compositions begin to produce an artificial tan. Depending on a number of factors, including formulation type, the resulting artificial tan continues to darken for a period of time and then begins to fade (due, in large part, to normal exfoliation). DHA has been widely approved for cosmetic use by many health and safety regulatory agencies.

In the 1960's, with the introduction of Coppertone's “Quick Tan”, sunless tanning products (also known as self-tanners or auto-bronzers) became available to consumers. However, these early products had aesthetically unattractive results, including non-natural looking coloration (often orange). With time, advances in formulating, including the introduction of erythrulose to DHA-containing sunless tanning products, led to more cosmetically-acceptable results.

As the associations between unprotected exposure to ultraviolet radiation and adverse health consequences (e.g., photoaging, actinic keratoses, melanoma, basal and squamous cell carcinoma) were reported in the scientific literature, the demand for sunless tanning products has increased. However, as a general matter, currently available sunless tanners still suffer from numerous drawbacks including uneven coloration, streaking, delayed onset of tanning and premature fading.

To remedy one or more of these drawbacks, various solutions have been proposed in the prior art, including the inclusion within the self-tanning compositions of polymeric gelling agents comprising 2-acrylamido-2-methylpropanesulphonic acid (AMPS) moieties.

For instance, EP-0 604 249 teaches cosmetic compositions for coloring the skin containing, in a cosmetically acceptable medium, dihydroxyacetone and at least one thickening agent consisting of a crosslinked copolymer of acrylamide/2-acrylamido-2-methylpropanesulphonic acid which has been partially or totally neutralised.

WO 2002/055052 teaches cosmetic compositions for artificial skin tanning and/or browning comprising, in a cosmetically acceptable medium, a self-tanning agent and at least one amphiphilic polymer containing at least one monomer having ethylenic unsaturation with a sulphonic group (such as AMPS), in free form or partially or totally neutralised, and comprising at least one hydrophobic part.

EP-1 570 836 and US 2006/239942 both teach compositions comprising a self-tanning agent and a hydrosoluble or hydrodispersible polymer or copolymer such as a copolymer of AMPS with a water-soluble comonomer including vinylpyrrolidone, as sold by CLARIANT under the trade name ARISTOFLEX® AVC.

DE-102 21 811 discloses a composition wherein an auto-bronzing agent such as DHA is stabilized by a malt extract, the composition also preferably comprising ARISTOFLEX® AVC as a gelling agent.

While some prior art self tanners have been able to achieve a desired rheology, these same formulations have one, and often two further drawbacks (in addition to the limitations discussed above)—the presence of an unacceptable colored complex (typically brown) and/or an undesirable odor (typically having a note characteristic of burnt caramel). Without wishing to be bound by a theory, the inventors believe that the cause of the brown colored complex and/or the burnt caramel-like odor in these prior art products is the presence of an autobronzing reactive material such as the above-mentioned AMPS polymers and copolymers.

The compositions of the present invention meet the need for self-tanning compositions that achieve a desired rheology without an unacceptable colored complex and/or an undesirable odor. They also provide for a long-lasting moisturizing effect.

### DETAILED DESCRIPTION OF THE INVENTION

The present invention is directed to an auto-bronzing composition comprising (i) at least one auto-bronzing agent, (ii) at least one auto-bronzing reactive material (“ARM”) chosen from the group consisting of amphiphilic copolymers of AMPS with at least one hydrophobic comonomer and (iii) at least one emulsifier of Formula I:

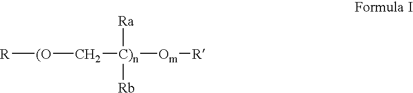

where
R is a C₁₂-C₂₀ alkyl or alkanoyl;

### Ra is H or OH;

### Rb is H, CH₃, CH₂OH or CH₂CH₃;

R′ is H or a C₁₂-C₂₀ alkanoyl;
n is an integer from 0 to 6000; and
m is 0 or 1.

It also pertains to the cosmetic use of an auto-bronzing composition as defined above for artificially tanning skin.

An “auto-bronzing agent” is a substance that reacts with the amine groups of amino acids, peptides and proteins in the stratum corneum to produce a colored (e.g., pigmented) complex having an appearance similar to that produced by melanogenesis.

In a preferred aspect of the present invention, the auto-bronzing agent is a “reducing sugar,” a carbohydrate with a hemiacetal group which, after undergoing a Maillard reaction, forms a polymeric carbonyl-amine compound. One particularly preferred reducing sugar is 1,3-dihydroxy-propan-2-one, also known as dihydroxyacetone (“DHA”). Another preferred reducing sugar is erythrulose.

In a most preferred aspect of this invention, the composition comprises DHA and a second reducing sugar selected from the group consisting of erythrulose, glucose, xylose, fructose, reose, ribose, arabinose, allose, tallose, altrose, mannose, galactose, lactose, sucrose, erthyrose and glyceraldehyde. A particularly preferred combination of auto-bronzing agents comprises or consists of DHA and erythrulose.

The amount of auto-bronzing agent(s) included in the composition of this invention may range from 1 to 15 wt % and preferably from 3 to 10 wt %, relative to the total weight of the composition.

The second component of the composition according to this invention is an ARM comprising an amphiphilic copolymer of 2-acrylamido 2-methylpropanesulfonic acid (AMPS) with at least one hydrophobic comonomer.

The AMPS monomer may be in a free form or in a neutralized or partially neutralized form, such as by an alkali metal hydroxide (sodium hydroxide or potassium hydroxide for instance) or by a nitrogen-containing base (such as mono-, di- or triethanolamine, aminomethylpropanediol, N-methylglucamine, basic amino acids such as arginine or lysine or ammonium hydroxide). In a most preferred embodiment, the 2-acrylamido 2-methylpropane-sulfonic acid is neutralized or partially neutralized by an ammonium hydroxide.

The hydrophobic comonomer may be chosen from the group consisting of ethylenically unsaturated monomers bearing at least one hydrophobic moiety which has from 6 to 50 carbon atoms and preferably from 6 to 22 carbon atoms.

Preferred hydrophobic comonomers may be chosen from acrylates and acrylamides of Formula II:

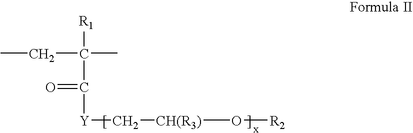

wherein:
R₁ and R₃, identical or different, are a hydrogen atom or a linear C₁-C₆ alkyl radical or a branched C₃-C₆ alkyl radical;

### Y is O or NH;

R₂ is a hydrocarbon radical comprising from 6 to 50 carbon atoms;
x is a number from 0 to 100.

According to a preferred embodiment of this invention, R₁ is a methyl group.

Preferably also, R₂ is a linear, branched or cyclic alkyl radical preferably comprising from 6 to 30 carbon atoms and more preferably from 12 to 22 carbon atoms, such as a n-dodecyl or a n-behenyl radical.

R₃ is preferably H, so the hydrophobic comonomer comprises at least one oxyethylene chain. As an alternative, R₃ may be a methyl group, so the hydrophobic comonomer comprises at least one oxypropylene chain.

Moreover, x is preferably at least 1 and may range from 3 to 100, preferably from 3 to 50 and more preferably from 7 to 25. Most preferably, x is 25. In a preferred embodiment, y is NH.

The resulting amphiphilic copolymers may be crosslinked or non-crosslinked. They are preferably crosslinked. They may comprise other hydrophilic or hydrophobic comonomers, such as an acrylamide comonomer.

Preferred amphiphilic copolymers according to this invention are the following:
those identified under the INCl name Ammonium Acryloyldimethyltaurate/Beheneth-25 Methacrylate Crosspolymer, sold for instance under the trade name Aristoflex® HMB by CLARIANT;
copolymers having from 15 to 60 weight percent of AMPS moieties and from 40 to 85 weight percent of (C₈-C₁₆)alkyl(meth)acrylamide or (C₈-C₁₆)alkyl(meth)acrylate moieties, such as described in EP-0 750 899;
terpolymers having from 10 to 90 mol percent of acrylamide moieties, from 0.1 to 10 mol percent of AMPS moieties and from 5 to 80 mol percent of n-(C₆-C₁₈)alkylacrylamide, as described for instance in U.S. Pat. No. 5,089,578.

These copolymers may be prepared according to typical processes of radical polymerization, for instance in tert-butanol, as described in WO 02/055052.

The amount of amphiphilic copolymer included in the composition of this invention may range from 0.05 to 5 wt % and preferably from 0 μl to 1 wt %, relative to the total weight of the composition.

Additional ARMs can be added to the compositions of the present invention, which are different from the AMPS-based ARM described above.

These ARMs are materials that, when added at a concentration of from about 0.1% to 2% to an aqueous solution containing 3.5% DHA and then stored at 45° C. for a period of four weeks, produce a calorimetric change, ΔE, of a least two units greater than a similar solution without the ARM based on the three-dimensional color coordinate system developed by the Commission Internationale d'Eclairage. More particularly, E is defined by the equation E=[(L*)²+(a*)²+(b*)²]^{1/2} where
L* is a value on the pale-dark axis;
a* is a value on the green-red axis; and
b* is a value on the yellow-blue axis.
   ΔE is the difference between E₁ (chromaticity value) after storage for four weeks at 45° C. and E₂ (chromaticity prior to elevated temperature storage) and is expressed by the equation:

ΔE=[(L*₁−L*₂)²+(a*₁−a*₂)²+(b*₁−b*₂)²]^{1/2}

The calorimetric changes, L*, a* and b* can be measured using a Minolta Spectrophotometer CM3600D.

In one aspect of the present invention, the additional ARM is a polymer.

In a preferred embodiment, the additional polymeric ARM comprises a nitrogen-containing monomer. In a particularly preferred embodiment, the additional polymeric ARM is selected from the group consisting of homo- and copolymers of (meth)acrylamides such as AMPS, ethyleneimines, imides, polyamides, pyrrolidines, polyquaternium compounds and muccopolysaccharides.

Preferred examples of additional ARMs suitable for use in the present invention include partially or totally neutralized homopolymers of AMPS and copolymers of AMPS with at least one water-soluble comonomer as described in US 2006/0239942, the content of which is incorporated herein by reference. Examples of such comonomers are (meth)acrylamide, maleic anhydride, N-vinyllactams such as N-vinylpyrrolidone, vinyl alcohol and (hydroxy)C₁-C₆-alkyl(meth)acrylates. Among these homo- and copolymers, mention can be made of those identified by the following INCI names: Ammonium Acryloyldimethyltaurate Vinylpyrrolidone Copolymer (sold for instance under the tradename Aristoflex® AVC by CLARIANT); Polyacrylamide/C₁₃-C₁₄ Isoparaffin/Laureth-7 (sold for instance under the trade names Sepigel® 305 and Simulgel® 600 by SEPPIC); Hydroxyethylacrylate/Sodium Acryloyldimethyltaurate copolymer (sold for instance under the trade name Simulgel® NS by SEPPIC).

In other embodiments, the additional ARM may be a polyethyleneimine. Non-limiting examples of polyethyleneimines suitable for use in the present invention include PEI-35, PEI-1500 and PEI-250, each commercially available from BASF under the tradenames Lupasol® G-35, Lupasol® PS and Lupasol® Waterfree.

In another embodiment, the additional ARM may be a polyimide. A non-limiting example of a polyimide suitable for use in the present invention is Polyimide-1 (INCI name: Isobutylene/dimethylaminopropyl maleimide/ethoxylated maleimide/maleic acid copolymer) such as sold under the tradename Aquaflex® XL30 by International Specialty Products.

In another embodiment, the additional ARM may be a polyquaternium compound. Non-limiting examples of polyquaternium compounds suitable for use in the present invention are: Polyquaternium-32 (such as sold as a mixture containing Mineral Oil and PPG-1 Trideceth-6 under the tradename Salcare® SC92 by CIBA Specialty Chemicals); Polyquaternium-37 (sold for instance as a mixture containing either Mineral Oil or Propylene Glycol Dicaprylate/Dicaprate and PPG-1 Trideceth-6 under the tradenames Salcare® SC 95 and SC 96 respectively by CIBA Specialty Chemicals); Polyquaternium-55 (sold for instance under the tradename Styleze® W-10 by International Specialty Products); Polyquaternium-28 (sold for instance under the tradename Gafquat® HS-100 by International Specialty Products).

In another aspect of the present invention, the additional ARM is a mucopolysaccharide having a nitrogen-containing group. Non-limiting examples of mucopolysaccharides having a nitrogen-containing group suitable for use in the present invention are chitosan and hyaluronan.

Additional ARMs not having a nitrogen-containing monomer may also be used in the auto-bronzing compositions of the present invention. Non-limiting examples of ARMs not having a nitrogen-containing monomer include sulfonated alkoxycellulose derivatives (such as sold under the tradename Poiz® 310 by KAO Corp.), polyacrylates (such as sold under the trade name Carbopol® by NOVEON, Inc.), polymethacrylates (such as sold under the trade name Diaformer® Z by CLARIANT Corp.), carageenans (such as sold under the trade name Viscarin® by FMC Biopolymer), alginates (such as sold under the trade name Protanal® by FMC BIOPOLYMERS), guars (such as sold under the trade name Jaguar® by RHODIA), xanthan (such as sold under the trade name Keltrol® by CP KELCO), sclerotium (such as sold under the trade name Amigel® by ALBAN MULLER Intl.) and gellans (such as sold under the trade name Kelcogel® by CP KELCO).

In a particular preferred embodiment of this invention, the composition comprises an additional ARM which is Aristoflex® AVC from CLARIANT.

The amount of additional ARM included in the composition of this invention may range from 0.1 to 3 wt % and preferably from 0.5 to 1 wt %, relative to the total weight of the composition.

The third component used in the compositions of the present invention is a specific emulsifier of Formula I

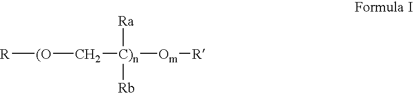

where
R is a C₁₂-C₂₀ alkyl or alkanoyl;

### Ra is H or OH;

### Rb is H, CH₃, CH₂OH or CH₂CH₃;

R′ is H or a C₁₂-C₂₀ alkanoyl;
n is an integer from 0 to 6000; and
m is 0 or 1.

This emulsifier acts as an ARM-stabilizing agent, i.e. it prevents or minimizes the formation of an unacceptable colored complex (typically brown) and/or an undesirable odor (typically having a note characteristic of burnt caramel) between the above-mentioned ARMs and the auto-bronzing agent in case it is a reducing sugar. An “unacceptable colored complex” is one that produces a ΔE of greater than about 25 after being stored at 45° C. for a period of eight weeks in an auto-bronzing composition comprised of one or more reducing sugars. In a preferred embodiment, an “unacceptable colored complex” is one that produces a ΔE of greater than about 25 after being stored at 45° C. for a period of eight weeks in an auto-bronzing composition comprised of DHA and erythrulose.

In a preferred embodiment of this invention
R is a C₁₂-C₂₀ linear alkanoyl such as a stearoyl group, and/or
R′ is H, and/or
Ra is H, and/or
Rb is H or CH₂OH, and/or
n is not zero, and/or
m is zero.

In a most preferred embodiment, the composition of this invention includes a blend of two emulsifiers according to Formula I, where the first emulsifier preferably corresponds to the formula R=n-stearoyl (C₁₈); R′=H; Ra=Rb=H; n=100; m=0, and the second emulsifier preferably corresponds to the formula R=n-stearoyl (C₁₈); Ra=H; Rb=CH₂OH; R′=H; n=1; m=0.

These emulsifiers are known under the INCI names PEG-100 stearate and glyceryl stearate, respectively, and sold for instance as a mixture under the trade names Simulsol® 165 by SEPPIC or Arlacel® 165 by UNIQUEMA.

In another embodiment of this invention:
R is a C₁₂-C₂₀ linear alkyl such as a stearyl group, and/or
R′ is H, and/or
Ra=Rb=H, and/or
n is 2 or 21, and/or
m is zero.

As an alternative, the composition may include a blend of two emulsifiers according to Formula I, where the first emulsifier corresponds to the formula R=n-stearyl (C₁₈); Ra=Rb=R′=H; n=2; m=0, and the second emulsifier corresponds to the formula R=n-stearyl (C₁₈); Ra=Rb=R′=H; n=21; m=0.

These emulsifiers are known under the INCI names steareth-2 and steareth-21, respectively, and sold for instance under the trade names Brij® 72 and Brij® 721 by UNIQUEMA.

The amount of emulsifier(s) of Formula I included in the composition of this invention may range from 1 to 10 wt % and preferably from 2 to 5 wt %, relative to the total weight of the composition.

At least one co-emulsifier such as cetyl alcohol, stearyl alcohol or cetearyl alcohol is further preferably included in the composition.

At least one sunscreen agent may also be used in the auto-bronzing compositions of the present invention. These include the sunscreens currently listed by the US Food and Drug Administration in the Sunscreen Drug Products for Over-The-Counter Human Use Final Monograph published in 64 Federal Register pp. 27666-27693 (May 21, 1999). Other sunscreen active ingredients are accepted for use in countries outside the US and are also considered to be within the scope of the present invention.

The sunscreens may be organic and/or inorganic compounds. Organic compounds are preferred according to this invention. Among them, mention can be made of dibenzoylmethane derivatives such as butyl methoxydibenzoylmethane (Parsol® 1789 from HOFFMANN LA ROCHE), cinnamic acid derivatives such as ethylhexyl methoxycinnamate (Parsol® MCX from HOFFMANN LA ROCHE), salicylates, para-aminobenzoic acids, β-β′-diphenylacrylate derivatives, benzophenone derivatives, benzylidenecamphor derivatives such as terephtalylidene dicamphor sulphonic acid, phenylbenzimidazole derivatives, triazine derivatives, phenylbenzotriazole derivatives, anthranilic derivatives, all of which may be coated or encapsulated. These sunscreens are all UV photoprotective agents well known per se.

In addition, the composition of the present invention may suitably contain various active agents which may be chosen from the group consisting of:
antioxidants, such as ascorbic acid and its salts, esters and ethers, including ascorbyl palmitate, ascorbyl tetraisopalmitate, ascorbyl glucoside, magnesium ascorbyl phosphate, sodium ascorbyl phosphate and ascorbyl sorbate; tocopherol and, its derivatives, such as tocopheryl acetate, tocopheryl sorbate and others esters of tocopherol; BHT and BHA; and plant extracts, for instance from Chondrus cripsus, Lilium candidum, Rhodiola, Thermus thermophilus, mate leaf, oak wood, kayu rapet bark, sakura leaves and ylang ylang leaves;
anti-ageing agents, such as Pisum sativum extracts, Zingiber officinale extracts, Centella asiatica extracts, methylsilanol derivatives such as methylsilanol mannuronate, hydrolyzed cucurbita pepo seedcake, Scenedesmus extract;
anti-pollution agents such as Moringa pterygosperma seed extracts;
keratolytic agents, such as α-hydroxyacids (for instance, glycolic, lactic, citric, malic, mandelic or tartaric acid) and β-hydroxyacids (for instance, salicylic acid), and their esters, including C₁₂₋₁₃ alkyl lactate, and plant extracts containing these hydroxyacids, such as Hibiscus sabdriffa extracts;
moisturizers, including polyols such as glycerol and propylene glycol; plant extracts such as Castanea sativa extracts, Polyanthes tuberosa polysaccharides and Argania spinosa kernel oil; homo- and copolymers of 2-methacryloyloxyethylphosphorylcholine, such as Lipidure® HM and Lipidure® PBM from NOF; saccharides such as glucose, fructose, mannose or trehalose; glycosaminoglycanes and their derivatives such as hyaluronic acid, sodium hyaluronate and acetylated hyaluronic acid; panthenol; allantoin; aloe vera; glucosamine; citric acid; urea and its derivatives and ceramides;
emollients such as glyceryl polymethacrylate, octyldodecyl neopentanoate, jojoba esters, shea butter, C₁₂₋₁₅ alkyl benzoate and silicon oils such as polydimethylsiloxanes (dimethicones);
anti-inflammatory agents, such as bisabolol, allantoin, tranexamic acid, sulfur oxide and its derivatives, chondroitin sulfate, glycyrrhizinic acid and its derivatives such as glycyrrhizinates
and their mixtures.

This composition may be in any form suitable for a topical application onto skin, such as an aqueous gel, an oil-in-water (O/W) or a water-in-oil (W/O) emulsion or a multiple emulsion (O/W/O or W/O/W), for instance. The composition according to this invention is preferably an oil-in-water emulsion.

In one embodiment, the overall pH of the auto-bronzing composition may be from about 2.5 to less than 7.0, preferably from about 3.0 to about 5.0.

### EXAMPLES

The following examples are further illustrative of the present invention. The components and specific ingredients are presented as being typical, and various modifications can be derived in view of the foregoing disclosure within the scope of the invention. All percentages, ratios and proportions herein are by weight, unless otherwise specified. All temperatures are in degrees Celsius unless otherwise specified.

### Example 1

### Auto-Bronzing Composition

| | |
|---|---|
| Ingredient | % wt/wt |

| | | |
|---|---|---|
| 101 | Demineralized Water | qs |
| 102 | Ammonium Acryloyldimethyltaurate/ | 0.20 |
| | Beheneth-25 Methacrylate Copolymer¹ | |
| 103 | Ammonium Acryloyldimethyltaurate/ | 0.30 |
| | VP Copolymer² | |
| 104 | Preservatives³ | 2.20 |
| 201 | C₁₂₋₁₅ Alkyl benzoate⁴ | 5.00 |
| 202 | Dimethicone⁵ | 2.50 |
| 203 | Cetearyl Alcohol⁶ | 0.75 |
| 204 | PEG-100 stearate and glyceryl stearate⁷ | 3.00 |
| 301 | Demineralized Water | 7.00 |
| 302 | Dihydroxyacetone USP | 7.00 |
| 303 | Erythrulose | 2.00 |

¹Commercially available as Aristoflex HMB from Clariant Corp. (Mount Holly, NC).
²Commercially available as Aristoflex AVC from Clariant Corp. (Mount Holly, NC).
³Commercially available as Elestab 388 from Laboratoires Serobiologiques (Pulnoy, France)
⁴Commercially available as Finsolv TN from Finetex (Elmwood Park, NJ)
⁵Commercially available as DC 200 Fluid 100 cSt (Dow Corning Corp., Midland, MI).
⁶Commercially available as Lanette O (Cognis, Ambler, PA).
⁷Commercially available as Simulsol 165 from SEPPIC (Paris, France).

A 500 gram batch comprising the ingredients listed above was prepared according to the following procedure.

Using a 1 Liter Greerco Homogenizer, into Main Vessel add 101-103. Begin slow speed homogenization until uniform. Add 104 to Main Vessel. Continue mixing until uniform. Heat to 70-75° C. and continue mixing. In Side Vessel A, pre-mix 201-204 at 75-80° C. Mix until all wax is completely dissolved. Continue mixing, maintain temperature and hold for phase combination. Add Side Vessel A to Main Vessel. Mix until uniform. Check microscope for particle size and tight border. Cool to 40° C. In Side Vessel B, pre-mix 301-303 at 25° C. Mix until uniform/clear and powder is completely dissolved. Add to the Main Vessel. Mix until uniform. Optionally, add a pH adjusting agent known to persons of skill in the art, q.s. until a pH of from 4.0 to 4.5. Mix until uniform.

### Example 2

### Evaluation of DHA and Color Stability

An auto-bronzing composition containing the following ingredients was prepared as described in Example 1, with the following modifications: ingredients 101 and 102 were added to a Main Vessel and mixed until uniform thereafter, ingredient 103 was added to the main vessel.

| Ingredient | | % wt/wt |
|---|---|---|
| 101 | Demineralized Water | qs |
| 102 | Ammonium Acryloyldimethyltaurate/ | 0.50 |
| | Beheneth-25 Methacrylate Copolymer¹ | |
| 103 | Preservatives³ | 2.20 |
| 201 | C₁₂₋₁₅ Alkyl benzoate⁴ | 5.00 |
| 202 | Dimethicone⁵ | 2.50 |
| 203 | Cetearyl Alcohol⁶ | 0.75 |
| 204 | PEG-100 stearate and glyceryl stearate⁷ | 3.00 |
| 301 | Demineralized Water | 7.00 |
| 302 | Dihydroxyacetone USP | 7.00 |
| 303 | Erythrulose | 2.00 |

It is well known and taught in the art that it is not recommended to have nitrogen containing ingredients in formulations containing DHA as there presence will cause rapid degradation of DHA. Surprisingly and unexpectedly, the content of DHA in this formulation is found to degrade less than about 15% after storage at 45° C. for six weeks. More specifically, a loss of 13.2% DHA is observed when assayed using an AGILENT 1100 Series HPLC.

Also, surprisingly and unexpectedly, this formulation is observed to exhibit a ΔE of less than 25% after eight weeks at 45° C. ΔE is measured using a Minolta Spectrophotometer CM-3600d and a calorimeter from Datacolor International Model SF 600 Plus. In contrast, in compositions identical to this formulation in all respects with the exception of the ARM-stabilizing agent (i.e. with the exception of emulsifier 204), ΔE values of greater than 70% are observed.

### Example 3

### In Vivo Evaluation

An auto-bronzing composition having the following ingredients was prepared in a similar way as previously described.

| | |
|---|---|
| Demineralized Water | qsp 100 |
| Propylene glycol | 3.00 |
| Glycerine | 5.00 |
| Sclerotium gum | 0.50 |
| Ammonium Acryloyldimethyltaurate/ | 0.20 |
| Beheneth-25 Methacrylate Copolymer¹ | |
| Ammonium Acryloyldimethyltaurate/ | 0.30 |
| VP Copolymer² | |
| Preservatives | 1.00 |
| PEG-100 stearate and glyceryl stearate⁷ | 4.00 |
| C₁₂₋₁₅ Alkyl benzoate⁴ | 5.00 |
| Dimethicone⁵ | 2.50 |
| Cetearyl Alcohol⁶ | 1.00 |
| Dihydroxyacetone USP | 7.00 |
| Erythrulose | 2.00 |
| Plant extracts | 2.05 |
| Perfume | qs |

This composition was applied in a single standard application to the forearm of 20 female volunteers presenting dry skin type on the forearms. Corneometric measurements were made at To, T2h, T4h and T8h. The moisturizing gain was 58.3% after 2 hours, 50.4% after 4 hours and still 30.1% after 8 hours, which demonstrates the long-lasting moisturizing effect of the compositions according to this invention. This is surprising since it is well-known in the art that DHA-containing compositions are drying to the skin.

Moreover, the above composition was tested by a panel of 10 people on one leg, in comparison with their usual auto-bronzing products applied on the other leg, using a self-evaluation questionnaire for the following consumer benefits:
Quicker onset of tan
More natural tan
More uniform tan
More intense tan
Longer lasting tan
Equal or better perfume

It was observed that
after a single application of the composition to the forearm, the color fully developed within 3 hours with the inventive composition, whereas it was only attained after 4 hours with usual products;
after a single application to the legs:
   the inventive composition was found to have tanned the skin like after a holiday week by 90% of the volunteers (whereas only 40% of the volunteers felt so regarding their usual product),
   90 to 100% of the volunteers found that the inventive composition allowed to dress quickly, provided a natural tan, a uniform color and a perfume at least as pleasant as their usual product;
after two consecutive applications to the legs on two consecutive days, the inventive composition was found to have tanned the skin like after a holiday week by 100% of the volunteers (whereas only 40% of the volunteers felt so regarding their usual product),
until 10 days after these two consecutive applications, the tan was said to be 100% more intense with the inventive composition than with the usual products;
until 10 days after these two consecutive applications, the color provided by the inventive composition was said to be still visible by 100% of all the volunteers, whereas only 40% of the volunteers felt so 10 days after the applications of their usual products; and
until 10 days after these two consecutive applications, the color provided by the inventive composition was still found to be uniform by 90% of the volunteers, whereas only 70% of the volunteers felt so 10 days after the applications of their usual products.

Thus, the compositions according to this invention offer a quicker, more uniform, more intense and more natural tan after application and this effect lasts at least ten days after application, which is not always the case with other sun-bronzing compositions currently on the market. Moreover, 90-100% of the volunteers felt that the inventive compositions are similar or even more pleasant than that of other marketed auto-bronzing compositions.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

1. Auto-bronzing composition comprising (i) at least one auto-bronzing agent, (ii) at least one auto-bronzing reactive material (“ARM”) chosen from the group consisting of amphiphilic copolymers of AMPS with at least one hydrophobic comonomer and (iii) at least one emulsifier of Formula I: 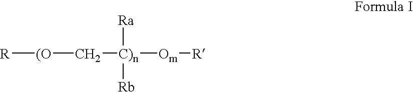
where
R is a Ci₂-C₂O alkyl or alkanoyl;
Ra is H or OH;
Rb is H, CH₃, CH₂OH or CH₂CH₃;
R′ is H or a C₁₂-C₂O alkanoyl;
n is an integer from 0 to 6000; and
m is 0 or 1.

2. Auto-bronzing composition according to claim 1, characterized in that said auto-bronzing agent is dihydroxyacetone (“DHA”).

3. Auto-bronzing composition according to claim 2, characterized in that the composition further comprises a reducing sugar selected from the group consisting of
erythrulose, glucose, xylose, fructose, reose, ribose, arabinose, allose, tallose, altrose, mannose, galactose, lactose, sucrose, erthyrose and glyceraldehyde.

4. Auto-bronzing composition according to claim 3, characterized in that the reducing sugar is erythrulose.

5. Auto-bronzing composition according to claim 1, characterized in that the amount of auto-bronzing agent (s) ranges from 1 to 15 wt % and preferably from 3 to 10 wt %, relative to the total weight of the composition.

6. Auto-bronzing composition according to claim 1, characterized in that the 2-acrylamido 2-methylpropane-sulfonic acid is neutralized or partially neutralized by an ammonium hydroxide.

7. Auto-bronzing composition according to claim 1, characterized in that the hydrophobic comonomer is chosen from the group consisting of ethylenically unsaturated monomers bearing at least one hydrophobic moiety which has from 6 to 50 carbon atoms and preferably from 6 to 22 carbon atoms.

8. Auto-bronzing composition according to claim 1, characterized in that the hydrophobic comonomer is chosen from acrylates and acrylamides of Formula II: 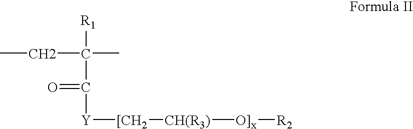
wherein: R₁ and R₃, identical or different, are a hydrogen atom or a linear C₁-C₆ alkyl radical or a branched C₃-C₆ alkyl radical;
Y is O or NH;
R₂ is a hydrocarbon radical comprising from 6 to 50 carbon atoms;
x is a number from 0 to 100.

9. Auto-bronzing composition according to claim 8, characterized in that R₁ is a methyl group.

10. Auto-bronzing composition according to claim 8, characterized in that R₂ is a linear, branched or cyclic alkyl radical comprising from 6 to 30 carbon atoms and preferably from 12 to 22 carbon atoms.

11. Auto-bronzing composition according to claim 10, characterized in that R₂ is a n-behenyl radical.

12. Auto-bronzing composition according to claim 8, characterized in that R₃ is H.

13. Auto-bronzing composition according to claim 8, characterized in that Y is NH.

14. Auto-bronzing composition according to claim 8, characterized in that x ranges from 3 to 100, preferably from 3 to 50 and more preferably from 7 to 25.

15. Auto-bronzing composition according to claim 14, characterized in that x is 25.

16. Auto-bronzing composition according to claim 8, characterized in that the amount of amphiphilic copolymer ranges from 0.05 to 5 wt % and preferably from 0.1 to 1 wt %, relative to the total weight of the composition.

17. Auto-bronzing composition according to claim 1, characterized in that the composition further comprises an additional polymeric ARM containing a nitrogen-containing monomer and selected from the group consisting of homo- and copolymers of (meth)acrylamides, ethyleneimines, imides, polyamides, pyrrolidines, polyquaternium compounds and mucopolysaccharides.

18. Auto-bronzing composition according to claim 17, characterized in that the additional polymeric ARM is selected from partially or totally neutralized homopolymers of AMPS and copolymers of AMPS with at least one water-soluble comonomer.

19. Auto-bronzing composition according to claim 18, characterized in that the water-soluble comonomer is selected from: (meth) acrylamide, maleic anhydride, N-vinyllactams, vinyl alcohol and (hydroxy)C₁-C₆-alkyl(meth)acrylates.

20. Auto-bronzing composition according to claim 19, characterized in that the water-soluble comonomer is N-vinylpyrrolidone.

21. Auto-bronzing composition according to claim 17, characterized in that the amount of additional polymeric ARM ranges from 0.1 to 3 wt % and preferably from 0.5 to 1 wt %, relative to the total weight of the composition.

22. Auto-bronzing composition according to claim 1, characterized in that:
R is a C₁₂-C₂₀ linear alkanoyl such as a stearoyl group, and/or
R′ is H, and/or
Ra is H, and/or
Rb is H or CH₂OH, and/or
n is not zero, and/or
m is zero.

23. Auto-bronzing composition according to claim 1, characterized in that the composition includes a blend of two emulsifiers according to Formula I, where the first emulsifier corresponds to the formula R=n-stearoyl (Ci₈); R′=H; Ra=Rb=H/n=100; m=0, and the second emulsifier corresponds to the formula R=n-stearoyl (C₁₈); Ra=H; Rb=CH₂CH; R′=H; n=1; m=0.

24. Auto-bronzing composition according to claim 1, characterized in that:
R is a C12-C20 linear alkyl such as a stearyl group, and/or
R′ is H, and/or·Ra=Rb=H, and/or
n is 2 or 21, and/or
m is zero.

25. Auto-bronzing composition according to claim 1, characterized in that the composition includes a blend of two emulsifiers according to Formula I, where the first emulsifier corresponds to the formula R=n-stearyl (C₁₈); Ra=Rb=R′=H; n=2; m=0, and the second emulsifier corresponds to the formula R=n-stearyl (C₁₈); Ra=Rb=R′=H; n=21; m=0.

26. Auto-bronzing composition according to claim 1, characterized in that the amount of emulsifier (s) of Formula I ranges from 1 to 10 wt % and preferably from 2 to 5 wt %, relative to the total weight of the composition.

27. Auto-bronzing composition according to claim 1, characterized in that at least one co-emulsifier chosen from cetyl alcohol, stearyl alcohol and cetearyl alcohol is included in the composition.

28. Auto-bronzing composition according to claim 1, characterized in that the composition further includes at least one sunscreen agent.

29. Auto-bronzing composition according to claim 1, characterized in that the composition is an oil-in-water emulsion.

30. Cosmetic use of an auto-bronzing composition according to claim 1 for artificially tanning skin.
